(19) United States
(12) Patent Application Publication
(10) Pub. No.: US 2003/0104033 A1
(43) Pub. Date: Jun. 5, 2003

(54) Enteral formulations

(76) Inventors: Chon-si Lai, Blacklick, OH (US); Bruce B. Blidner, Westerville, OH (US); David A. Deis, Westerville, OH (US); Paul W. Johns, Columbus, OH (US); Timothy W. Schenz, Powell, OH (US)

Correspondence Address: ROSS PRODUCTS DIVISION OF ABBOTT LABORATORIES, DEPARTMENT 108140-DS/1, 625 CLEVELAND AVENUE, COLUMBUS, OH, 43215-1724, US

(21) Appl. No.: 09/905,022

(22) Filed: Jul. 13, 2001

(51) Int. Cl. A61K 9/00; A61K 47/00

(52) U.S. Cl. 424/439; 424/400

(57) ABSTRACT

The present invention is directed to a new class of enteral formula containing an admixture of casinate and a stabilizing protein, which is either whey or vegetable. These new enteral formula exhibit significantly reduced creaming when compared to the enteral formula of the prior art in which caseinate was the sole protein source. The invention also includes a method of reducing creaming in enteral formula.

The present invention is directed to a new class of enteral formula having a protein system that contains a stabilizing protein and caseinate. These formula exhibit a reduced rate of creaming and an enhanced shelf life.

### BACKGROUND

Enteral formulas represent an important component of patient care in both acute care hospitals and long term care facilities (i.e. nursing homes). These formulas typically serve as the sole source of nutrition over an extended period of time. Accordingly, the formulas must contain significant amounts of protein, fat, minerals, electrolytes, etc., if they are to meet their primary goal of preventing malnutrition. These formulas are typically administered to the patient as a liquid, since the patient is generally incapable of consuming solid foods. While some patients are capable of drinking the formula, most patients receive these nutritionals via a nasogastric tube (NG tube or tube feeding).

Enteral formulas may be sold in one of two forms. The first is as a powder that is reconstituted immediately prior to administration by the nurse or dietician. The second is a ready-to-feed liquid (RTF) that is simply attached to the NG tube at the time of administration. In the United States, health care facilities overwhelming prefer the ready-to-feed formula in light of the shortages of trained medical personnel in many communities. Further, health care facilities expect these RTF formula to have a shelf life of at least 12 months. This expectation of long term stability has created a number of stability issues, some of which have only partially been solved.

These RTF formula contain substantial quantities of lipids, since lipids are required to avoid malnutrition. Therefore, these RTF formula are typically manufactured as oil-in-water emulsions. An emulsion is a stable admixture of two, or more, immiscible liquids, which are held in suspension by substances which are referred to as emulsifiers. Surfactants, which serve as emulsifiers, are routinely incorporated into enteral formula. Proteins and carbohydrate polymers are also capable of acting as emulsifiers and further serve to stabilize the formula. These multiple emulsifiers have not solved all of the stability problems associated with RTF formula.

One such problem is creaming. Creaming is a descriptive term for phase separation. Instead of having two immiscible layers in suspension, the lipid layer separates from the aqueous layer and floats to the top of the container. Creaming causes a number of problems.

One problem is the uneven, or incomplete, delivery of nutrients. Since the fat is at the top of the container, the patient receives the lipid calories as a bolus at the very end of the administration period, (which can be up to 24 hours). The separated fat layer often clings to the side of the bottle, as well as the administration set, resulting in the non-delivery of a substantial portion the lipid. If the fat remains in the NG tubing for an extended period between enteral feedings, it is possible for the lipid to harden and block the NG tube.

In addition to problems with the delivery of nutrients, the physical appearance of the enteral formula is negatively impacted by the phase separation. If the creaming is severe enough, it can actually cause the formula to resemble spoiled milk. Attempts have been made to solve this problem, but the solutions developed to date have not been adequate, especially for products having elevated caloric densities. Creaming is exacerbated in formulas having a caloric density greater than 1 kcal/ml. Caloric densities in this range are often used since it allows a patient's nutritional needs to be met in a volume of approximately 1 liter.

U.S. Pat. No. 5,700,513 to Mulchandani et al is directed to enhancing the physical stability of enteral formula. It teaches that iota carrageenan and cellulose derivatives will decrease creaming problems. U.S. Pat. No. 5,869,118 to Morris et al. is also directed to improving the stability of enteral formula. It teaches that gellan gum will reduce the incidence of creaming. U.S. Pat. No. 5,416,077 to Hwang et al teaches that iota carrageenan and kappa carrageenan will also reduce creaming. While these patents are a significant contribution to the art, their solutions have not been entirely adequate, especially in calorically dense nutritionals.

While a number of researchers have focused upon additives or stabilizers to reduce the incidence of creaming, the literature does not describe any attempt to evaluate protein sources and their impact upon creaming.

### SUMMARY OF THE INVENTION

In accordance with the present invention, it has been discovered that the incidence of creaming in enteral formula can be decreased by the utilization of a particular protein system. This protein system contains from about 40 to about 95 w/w % of caseinate and from about 5 to about 60 w/w % of a stabilizing protein, based upon the total protein content of the formula. The stabilizing protein is selected from the group consisting of vegetable protein and whey protein. The preferred stabilizing protein is soy.

Enteral formula utilizing this protein system will exhibit an absence, or a significant reduction in creaming, when compared to an enteral formula utilizing caseinate as the sole source of protein. This absence, or reduction, of creaming will be maintained for a period of at least 12 months. This finding was entirely unexpected. Caseinate has a long history of use in the dairy industry as an emulsifying protein. Caseinate is routinely used in oil-in water emulsions since it has desirable organoleptics, a desirable amino acid profile, and was thought to significantly enhance the stability of the emulsion. The inventor's finding that caseinate actually destabilizes the enteral formula by promoting phase separation was entirely unexpected.

Despite the destabilizing impact of the caseinate, the protein system should contain at least 40% of caseinate. The inventors have discovered that when the content of stabilizing protein is increased above 60%, the formulations become unstable. The protein precipitates from the emulsion, especially after thermal processing.

A further aspect of the invention is directed to a new class of enteral formula which utilize this protein system. These nutritionals comprise:

a) a protein system providing at least 16% of the total calories of said nutritional, in which said protein system contains;

i. a source of caseinate protein, present in the quantity of about 40 w/w % to about 95 w/w %, based upon the total protein content of the nutritional, and,

ii. a stabilizing protein selected from the group consisting of vegetable protein and whey protein, in which said stabilizing protein is present in the quantity of about 5 w/w % to about 60 w/w %, based upon the total protein content of the nutritional;

b) a source of fat, providing at least 25% of the total calories of said nutritional;

c) a source of carbohydrate, providing at least 30% of the total calories of said nutritional, and;

d) at least 8 grams of a source of fiber, per liter of said nutritional.

### DETAILED DESCRIPTION OF THE INVENTION

As used in this application:

a) the term “enteral formula”, “nutritional formula”, and “product” are being used interchangeably.

b) the term “total calories” refers to the total caloric content of a defined volume of the finished nutritional product (i.e. calories per liter).

c) Any reference to a numerical range in this application should be construed as an express disclosure of every number specifically contained within that range and of every subset of numbers contained within that range. Further, this range should be construed as providing support for a claim directed to any number, or subset of numbers in that range. For example, a disclosure of 1-10 should be construed as supporting a range of 2-8, 3-7, 5, 6, 1-9, 3.6-4.6, 3.5-9.9, 1.1-9.9, etc.

d) The term “total protein content of the formula” is based on the total kjeldahl nitrogen minus non-protein nitrogen

e) The term “RDIs” refers to a set of dietary references based on the Recommended Dietary Allowances (RDA) for essential vitamins and minerals.

The name “RDI” replaces the term “U.S. RDA” (Recommended Daily Allowances). Recommended Dietary Allowances (RDA) are the set of estimated nutrient allowances established by the National Academy of Sciences used as the basis for setting the U.S.RDAs. It is updated periodically to reflect current scientific knowledge.

The key to the present invention is the unique protein system described above. This protein system significantly reduces, or eliminates, phase separation in these oil-in-water emulsions and thus significantly minimizes the creaming problems described above. This protein system can be used in essentially any of the prior art enteral formulas marketed to date, by merely substituting the protein system of the invention for that of the prior art. This protein system can be used in enteral formula's designed for the general population or for populations suffering from a particular disease or injury.

For example, diabetics experience a sharp rise in blood glucose levels when fed traditional enteral formula. Therefore, specialized formulas have been developed for these patients. These formulas often contain relatively greater quantities of lipids in order to blunt the patients glycemic response. These formula often have significant creaming problems and thus can benefit from application of the protein system of this invention. Examples of such diabetic formula includes Glucerna®, which is marketed by Abbott Laboratories and Glytrol® which is marketed by Nestle.

Specialized formula have been designed for long term care facilities where patients have a substantial risk of developing pressure ulcers due to their limited mobility. These formula often contain elevated quantities of caseinate to promote healing and thus suffer from significant creaming problems. Examples of such formulas include Jevity®, Jevity Plus®, Twocal®, Periative®, and NutriFocus®, all of which are marketed by Abbott Laboratories. Other examples include Probalance® which is marketed by Nestle and Ultracal® which is marketed by Mead Johnson.

The specific enteral formulas described above are only an attempt to illustrate the many potential applications to which the present invention can be applied. Those skilled in the art will readily recognize other classes of formula whose stability can be improved by the protein system of this invention.

As is well known to those skilled in the art, tube feeding formula typically serves as the sole source of nutrition. Therefore, it must contain protein, carbohydrate, lipids, vitamins, and minerals. These nutrients must be present in quantities sufficient to prevent malnutrition in a human, in a volume that can readily be consumed or administered in 24 hours. Typically, this entails a caloric requirement of 1000 calories to 3000 calories per day. These calories should be provided in a volume ranging from 1 to 2 liters.

One component of the formulas of this invention is the protein system. The protein system should provide at least 16% of the total calories of the nutritional. It can provide up to about 35% of total calories. In a further embodiment, it provides from about 16.5% to about 25% of the total calories of the nutritional, and more typically about 18-25% of total calories.

The protein system utilized in the present invention must contain at least two different types of protein. The first protein that must be present is the caseinate. Caseinate should be present in the formulation due to the stability problems described above. The inventors have surprisingly discovered that if the concentration of the stabilizing protein exceeds 60%, a different stability problem is encountered. At these concentrations, protein precipitates from the emulsion. This precipitation is exacerbated when the formula is thermally processed to achieve food grade sterility. Caseinate is the acid insoluble fraction of protein obtained from mammalian milk. Preferably, the caseinate is obtained from bovine, but it may be obtained from any mammal whose milk is routinely consumed by humans. Suitable types of caseinate include sodium caseinate, calcium caseinate, potassium caseinate, magnesium caseinate, lithium caseinate, etc. The caseinate is preferably intact. However, it may be slightly hydrolyzed. If a hydrolyzed source of caseinate is used, it should have a degree of hydrolysis (DH) of 10% or less. Degree of hydrolysis refers to the percentage of peptide bonds that are cleaved. This is described in greater detail, including methods for determining DH, by Adler-Nissen, in Journal of Agricultural Food Chemistry, 27/6 (1979) 1256-1262.

Caseinate is available from numerous commercial sources. For example, caseinates, and hydrolyzed caseinates, are available from New Zealand Milk Products of Harrisburg, Pa.

The quantity of caseinate contained within the protein system can vary, but the protein system should contain at least 40 w/w % of caseinate, based upon the total protein content of the formula. Caseinate content can run as high as 95 w/w %, based upon the total protein content. More typically, the caseinate will be present in a quantity ranging from about 60 to about 85% of and more typically from about 60 to about 80 w/w %, based upon total protein content.

The other component of the protein system is the stabilizing protein. The stabilizing protein should be a vegetable protein or whey protein. Vegetable protein is derived from any vegetable source (i.e. non-animal) Examples of suitable vegetable proteins include soy, corn, potato, rice and pea. The vegetable protein is preferably intact, but it may be slightly hydrolyzed. It should not possess a DH of greater than about 10%. The most preferred vegetable protein is soy. The soy may be present as either soy protein concentrate or soy protein isolate.

The stabilizing protein may also be whey protein. Whey protein is the acid soluble fraction of a protein obtained from mammalian milk. Preferably, the whey is obtained from bovine, but it may be obtained from any mammal whose milk is routinely consumed by humans. The whey is preferably intact, but may have a DH of 10% or less.

These stabilizing proteins are available from a number of commercial sources. For example, intact whey and hydrolyzed whey are available from New Zealand Milk Products of Harrisburg, Pa. Soy and hydrolyzed soy proteins are available from Protein Technologies International of Saint Louis, Mo. Pea protein is available from Feinkost Ingredients Company of Lodi, Ohio. Rice protein is available from California Natural Products of Lathrop, Calif. Corn protein is available from EnerGenetics Inc. of Keokuk, Iowa.

The stabilizing protein may be either whey or a vegetable protein. It may also be an admixture of whey and one or more vegetable proteins, or an admixture of different vegetable proteins. The quantity of stabilizing protein can vary widely, but will typically range from about 5 w/w % of the total protein content, up to about 60 w/w % of the total protein content. In a further embodiment, the stabilizing protein is present in the quantity of from about 15 to about 40 w/w % and more typically from about 20 to about 35 w/w % of the total protein content.

As is well known to those skilled in the art, isolates and concentrates of milk protein are commercially available (hereinafter “isolates”) and may be incorporated into enteral formulas. These milk protein isolates contain both whey and caseinate, in varying amounts. These isolates may be utilized in the formulas of this invention to provide both the required caseinate and stabilizing protein. Theses isolates should be treated as if the whey and caseinate contained within the isolate were being incorporated separately, when determining if they meet the limitations of the claims. For example, 10 grams of milk protein isolate containing 70% caseinate and 30% whey; should be treated as if 7 grams of casinate and 3 grams of whey were added to the nutritional.

In addition to the caseinate and the stabilizing protein, the formula may optionally contain free amino acids, or small peptides, if the patient would benefit from such additives. For example, arginine promotes the healing of pressure ulcers and helps to maintain the integrity of the skin. Patients suffering from traumatic injuries may benefit from the presence of glutamine or peptides containing glutamine. Other amino acids or peptides whose presence may be beneficial include methionine. If amino acids or peptides are incorporated into the formula, their collective quantity should not exceed 20 w/w % of the total protein content, and more typically about 10 w/w %.

In addition to the protein, the formulas must contain lipids, or fats. Lipids provide energy and essential fatty acids and enhance the absorption of fat soluble vitamins. The quantity of lipid utilized in the formulas of this invention can vary widely. However, creaming is typically not a problem in formulas in which the fat content is below about 25% of total calories.

As a general guideline however, lipids should provide at least about 25% of the total calories of the formula and may provide up to about 60% of total calories. In a further embodiment, the lipid provides from about 30% to about 50% of total calories. The source of the lipids is not critical to the invention. Any lipid, or combination of lipids, that provides all essential fatty acids and that is suitable for human consumption may be utilized.

Examples of food grade lipids suitable for use in the formulas of this invention include soy oil, olive oil, marine oil, sunflower oil, high oleic sunflower oil, safflower oil, high oleic safflower oil, fractionated coconut oil, cottonseed oil, corn oil, canola oil, palm oil, palm kernel oil and mixtures thereof. Numerous commercial sources for these fats are readily available and known to one practicing the art. For example, soy and canola oils are available from Archer Daniels Midland of Decatur, Ill. Corn, coconut, palm and palm kernel oils are available from Premier Edible Oils Corporation of Portland, Organ. Fractionated coconut oil is available from Henkel Corporation of LaGrange, Ill. High oleic safflower and high oleic sunflower oils are available from SVO Specialty Products of Eastlake, Ohio. Marine oil is available from Mochida International of Tokyo, Japan. Olive oil is available from Anglia Oils of North Humberside, United Kingdom. Sunflower and cottonseed oils are available from Cargil of Minneapolis, Minn. Safflower oil is available from California Oils Corporation of Richmond, Calif.

In addition to these food grade oils, structured lipids may be incorporated into the nutritional if desired. Structured lipids are known in the art. A concise description of structured lipids can be found in INFORM, Vol. 8, No. 10, page 1004, entitled Structured lipids allow fat tailoring (October 1997). Also see U.S. Pat. No. 4,871,768 which is hereby incorporated by reference. Structured lipids are predominantly triacylglycerols containing mixtures of medium and long chain fatty acids on the same glycerol nucleus. Structured lipids and their use in enteral formula are also described in U.S. Pat. Nos. 6,194,37 and 6,160,007, the contents of which are hereby incorporated by reference.

The nutritionals of this invention will also contain a source of carbohydrates. Carbohydrates are an important energy source for the patient as they are readily absorbed and utilized. They are the preferred fuel for the brain and red blood cells. The quantity of carbohydrate that may be utilized can vary widely. Typically, sufficient carbohydrates will be utilized to provide at least 25% of total calories. Carbohydrates may provide up to bout 60% of total calories. Typically, carbohydrates will provide from about 25% to about 55% of total calories.

The carbohydrates that may be used in these formula can vary widely. Any carbohydrate source typically used in the industry may be used. Examples of suitable carbohydrates that may be utilized include hydrolyzed corn starch, maltodextrin, glucose polymers, sucrose, corn syrup solids, glucose, fructose, lactose, high fructose corn syrup and fructooligosaccharides.

Specialized carbohydrate blends have been designed for diabetics to help moderate their blood glucose levels. Examples of such carbohydrate blends are described in U.S. Pat. No. 4,921,877 to Cashmere et al., U.S. Pat. No. 5,776,887 to Wibert et al., U.S. Pat. No. 5,292,723 to Audry et al. and U.S. Pat. No. 5,470,839 to Laughlin et al, the contents of which are all incorporated by reference. Any of these carbohydrate blends may be utilized in the nutritionals of this invention.

Along with a source of carbohydrate, the formulas of this invention will also contain a source of fiber. The exact impact of fiber on creaming is not understood, but the most significant creaming problems noted by the inventors, have occurred in formulas containing significant quantities of fiber. Dietary fiber, as used herein and in the claims, is understood to be all of the components of a food that are not broken down by enzymes in the human digestive tract to small molecules which are absorbed into the bloodstream. These food components are mostly celluloses, hemicelluloses, pectin, gums, mucilages, and lignins. Fibers differ significantly in their chemical composition and physical structure and therefore their physiological functions.

The properties of fibers (or fiber systems) that impact on physiological function are solubility and fermentability. With regard to solubility, fiber can be divided into soluble and insoluble types based on the fiber's capacity to be solubilized in a buffer solution at a defined pH. Fiber sources differ in the amount of soluble and insoluble fiber they contain. As used herein and in the claims “soluble” and “insoluble” dietary fiber is determined using American Association of Cereal Chemists (AACC) Method 32-07. As used herein and in the claims, “total dietary fiber” or “dietary fiber” is understood to be the sum of the soluble and insoluble fibers determined by AACC Method 32-07 and wherein by weight, at least 70% of the fiber source comprises dietary fiber. As used herein and in the claims a “soluble” dietary fiber source is a fiber source in which at least 60% of the dietary fiber is soluble dietary fiber as determined by AACC Method 32-07, and an “insoluble” dietary fiber source is a fiber source in which at least 60% of the total dietary fiber is insoluble dietary fiber as determined by AACC Method 32-07.

Representative of soluble dietary fiber sources are gum arabic, sodium carboxymethyl cellulose, guar gum, citrus pectin, low and high methoxy pectin, oat and barley glucans, carrageenan and psyllium. Numerous commercial sources of soluble dietary fibers are available. For example, gum arabic, hydrolyzed carboxymethyl cellulose, guar gum, pectin and the low and high methoxy pectins are available from TIC Gums, Inc. of Belcamp, Md. The oat and barley glucans are available from Mountain Lake Specialty Ingredients, Inc. of Omaha, Nebr. Psyllium is available from the Meer Corporation of North Bergen, N.J. while the carrageenan is available from FMC Corporation of Philadelphia, Pa.

Representative of the insoluble dietary fibers are oat hull fiber, pea hull fiber, soy hull fiber, soy cotyledon fiber, sugar beet fiber, cellulose and corn bran. Numerous sources for the insoluble dietary fibers are also available. For example, the corn bran is available from Quaker Oats of Chicago, Ill.; oat hull fiber from Canadian Harvest of Cambridge, Minn.; pea hull fiber from Woodstone Foods of Winnipeg, Canada; soy hull fiber and oat hull fiber from The Fibrad Group of LaVale, Md.; soy cotyledon fiber from Protein Technologies International of St. Louis, Mo.; sugar beet fiber from Delta Fiber Foods of Minneapolis, Minn. and cellulose from the James River Corp. of Saddle Brook, N.J.

A more detailed discussion of and fibers and their incorporation into formula may be found in U.S. Pat. No. 5,085,883 issued to Garleb et al, which is hereby incorporated by reference.

The quantity of fiber utilized in the formulas can vary, but the formula should contain at least 8 grams of fiber per liter. The nutritional will typically contain from about 10 to about 35 grams per liter of fiber. Most preferably, the fiber will be present in a quantity raning from about 10 to about 20 grams per liter. The particular type of fiber that is utilized is not critical. Any fiber suitable for human consumption and that is stable in the matrix of a nutritional formula may be utilized. In addition to fiber, the nutritionals may also contain oligosaccharies such as fructooligosaccharies (FOS) or glucooligosacchairdes (GOS). Oligosaccharides are rapidly and extensively fermented to short chain fatty acids by anaerobic microorganisms that inhabit the large bowel. These oligosaccharides are preferential energy sources for most Bifidobacteum species, but are not utilized by potentially pathogenic organisms such as Clostridium perfingens, C. difficile, or E. coli.

The nutritionals of this invention will contain sufficient vitamins and minerals to meet all of the relevant RDI's. Those skilled in the art recognize that nutritionals often need to be over fortified with certain vitamins and minerals to insure that they meet the RDI's over the shelf life of the product. These same individuals also recognize that certain micronutrients may have potential benefits for people depending upon any underlying illness or disease that the patient is afflicted with. For example, diabetics benefit from nutrients such as chromium, carnitine, taurine and vitamin E. Modifying vitamin and mineral content to meet all RDI's, as well as to meet the needs of a particular population is well within the skills of one skilled in the art.

An example of the vitamin and mineral system for a formula of this invention typically comprises at least 100% of the RDI for the vitamins A, B₁, B₂, B₆, B₁₂, C, D, E, K, beta-carotene, Biotin, Folic Acid, Pantothenic Acid, Niacin, and Choline; the minerals calcium, magnesium, potassium, sodium, phosphorous, and chloride; the trace minerals iron, zinc, manganese, copper, and iodine; the ultra trace minerals chromium, molybdenum, selenium; and the conditionally essential nutrients m-inositol, carnitine and taurine, in a volume ranging from about 1 liter to about 2 liters.

As is well known to those skilled in the art, the caloric density of enteral formula can vary. Creaming becomes more problematic as the caloric density of the formulation increases. The stabilizing protein system described above is especially applicable to formula with caloric densities ranging between about 1 kilocalorie (kcal)/milliliter and 2.5 kcal/ml. It is especially applicable for formula having a caloric density between 1.2 kcal/ml and 2.0 kcal/ml.

Artificial sweeteners may also be added to the nutritional formula to enhance the organoleptic quality of the formula. Examples of suitable artificial sweeteners include saccharine, aspartame, acesulfame K and sucralose. The nutritional products of the present invention may optionally include a flavoring and/or color to provide the nutritional products with an appealing appearance and an acceptable taste for oral consumption. Examples of useful flavorings typically include, for example, strawberry, peach, butter pecan, chocolate, banana, raspberry, orange, blueberry and vanilla.

The nutritional products of this invention can be manufactured using techniques well known to those skilled in the art. While manufacturing variations are certainly well known to those skilled in the nutritional formulation arts, a few of the manufacturing techniques are described in detail in the Examples. Generally speaking an oil and fiber blend is prepared containing all oils, any emulsifier, fiber and the fat soluble vitamins. Three more slurries (carbohydrate and two protein) are prepared separately by mixing the carbohydrate and minerals together and the protein in water. The slurries are then mixed together with the oil blend. The resulting mixture is homogenized, heat processed, standardized with water soluble vitamins, flavored, and terminally sterilized. The formula may then be packaged in any form that is desirable to the consumer or health care practitioner.

The following Examples are being presented in order to further illustrate the invention. They should not be construed as limiting the invention in any manner. The specific embodiments illustrated by these examples will illustrate to those skilled in the art the wide ranging applicability of the stabilizing protein system of this invention.

### EXAMPLE I

Two 1.06 Kcal/ml fiber containing ready-to-feed tube feed products with 16.7% protein calories, 29% fat calories and 53.3% carbohydrate calories were manufactured in a pilot plant facility using multiple lots of protein and fiber ingredients. Table 1 and 2 showed the BOMs of a 1000 lb batch of the control (100% caseinates) and 20% SPI formulation.

TABLE 1
BOM of 100% caseinates formulation

| | Amount per 1000 |
|---|---|
| Ingredient | lbs of products |
| Water | 761 |
| Maltodextrin M-100 | 135 |
| Sodium caseinates | 35.9 |
| High Oleic Safflower Oil | 9.42 |
| CANOLA OIL | 9.21 |
| Fructooligossachrides | 7.12 |
| Medium Chain Triglyceride Oil | 6.28 |
| Corn oil | 6.28 |
| Calcium CASEINATE | 5.46 |
| OAT FIBER | 5.02 |
| Soy Fiber | 4.22 |
| Tricalcium phosphate | 2.28 |
| GUM ARABIC | 1.99 |
| Diacetyltartaric Acid Esters | 1.65 |
| SODIUM CITRATE | 1.60 |
| Potassium Chloride | 1.43 |
| MgHPO4 | 1.43 |
| POTASIUM CITRATE | 1.25 |
| Carboxymethyl Cellulose | 0.903 |
| CHOLINE CHOLORIDE | 0.507 |
| 45% KOH | 0.307 |
| ASCORBIC ACID | 0.284 |
| UTM/TM | 0.214 |
| Magnesium Chloride | 0.214 |
| CARNITINE | 0.154 |
| TAURINE | 0.146 |
| VITAMIN PREMIX | 0.0957 |
| Gellan Gum | 0.0500 |
| Vitamin DEK premix | 0.0459 |
| beta Carotene | 0.00712 |
| NaF | 0.003067 |
| POTASIUM IODIDE | 0.00015 |

TABLE 2
BOM of 20% SPI formulation

| | |
|---|---|
| Water | 762 |
| Maltodextrin M-100 | 135 |
| Na-caseinates | 28.7 |
| HOSO | 9.42 |
| CANOLA OIL | 9.21 |
| Soy Protein Isolate | 7.60 |
| FOS | 7.12 |
| MCT | 6.28 |
| Corn oil | 6.28 |
| Ca-CASEINATE | 5.46 |
| OAT FIBER | 5.02 |
| Soy Fiber | 4.22 |
| TCP | 2.28 |
| GUM ARABIC | 1.99 |
| Diacetyltartaric Acid Esters | 1.65 |
| SODIUM CITRATE | 1.60 |
| Potassium Chloride | 1.43 |
| MgHPO4 | 1.43 |
| POTASIUM CITRATE | 1.25 |
| Carboxymethyl Cellulose | 0.903 |
| CHOLINE CHOLORIDE | 0.507 |
| 45% KOH | 0.307 |
| ASCORBIC ACID | 0.284 |
| UTM/TM | 0.214 |
| Magnesium Chloride | 0.214 |
| CARNITINE | 0.154 |
| TAURINE | 0.146 |
| VITAMIN PREMIX | 0.0957 |
| Gellan Gum | 0.0500 |
| Vitamin DEK premix | 0.0459 |
| beta Carotene | 0.00712 |
| NaF | 0.003067 |
| POTASIUM IODIDE | 0.000150 |

Two protein-in-fat slurries are prepared by placing canola oil, high oleic safflower oil, and medium chain triglycerides oil to a tank and heat the oil blend to a temperature in the range of 140 to 150° F. Under agitation, the target amount of oil soluble vitamins and Panodan are added to oil blend. The soy protein isolate or sodium caseinates is then added to the oil blend.

The protein-in-water slurries are prepared by dispersing target weights of proteins in about 400 lbs of water and gradually heat the slurry to 130 to 140° F. under agitation.

A carbohydrate/mineral slurry is prepared by placing about 150 lbs of water in a kettle and heats the water to 130 to 150° F. Under agitation, add the target amounts of salts, fibers and maltodextrins. Hold the slurry at 130 to 150° F. until use.

A vitamin solution is prepared by dissolving the vitamins, carnitine, choline and taurine in about 26 lbs of water and the pH of the solution is adjusted to 6.5 to 10.5 using 45% KOH.

A blend is prepared by adding the carbohydrate slurry to the protein in water slurry under agitation. The protein-in-oil slurry is then added to the blend and the pH of the blend is adjusted to 6.6 to 6.8 using IN KOH. The blends are UHT and homogenized. The vitamin solution is then added to the homogenized blend and water is added to adjust the fat, protein and total solids level to the desired ranges. The standardized products are then filled in semi translucent plastic containers and retorted to achieve sterility.

The finished products are stored in upright position at room temperature and samples are delivered to physical testing laboratory to measure the thickness of the cream layer during shelf life testing (Table 3). The term “cream” describes a layer of viscous oily liquids floating on top the product and it only become visible after storage. The presence of a viscous cream layer in the ready-to-feed product renders the product less appealing. In addition, this cream layer tends to smear the neck area of the container after shaking and raises customer concern about product quality. Thus, the creaming defect is one of the important factors limiting product shelf life.

We found that the inclusion of SPI as part of the protein system delayed the onset of creaming (Table 3). There was no measurable creaming in the first 5 months of storage.

TABLE 3
Effect of inclusion of SPI on Cream Stability

| | 100% | | 20% SPI 1 | 20% SPI 2 | |
|---|---|---|---|---|---|
| | caseinates | 100% | (ingredient | (ingredient | |
| | (ingredient | caseinates | lot A) | Lot B) | 20% SPI 3 |
| | Lot A) | (ingredient | Thickness | Thickness | (Ingredient |
| Age of | Thickness | lot B) | of | of | lot C |
| product | of cream | Thickness of | cream | cream | Thickness of |
| (month) | (mm) | cream (mm) | (mm) | (mm) | cream (mm) |
| 0.00 | 0.00 | 0.00 | 0.00 | 0.00 | 0.00 |
| 3.00 | 0.00 | 0.00 | 0.00 | 0.00 | 0.00 |
| 5.00 | 8.00 | 7.00 | 0.00 | 0.00 | 0.00 |

We visually inspected the 7 months old samples after they were shaken using an invert bottle 3 second shaking. We noticed that inclusion of SPI significantly reduce the amount of cream sticking to the container.

### EXAMPLE 2

Three 1.2 Kcal/ml fiber containing ready-to-feed tube feed products with 18% protein calories, 29% fat calories and 53% carbohydrate calories were manufactured in a pilot plant facility using a procedure very similar what was described in example 1. Table 5, 6, and 7 showed the BOMs of a 1000 lb batch of the control (100% caseinates) and 20% SPI formulation.

TABLE 4
BOM of a 100% caseinate 1.2 Kcal/ml fiber containing product

| Ingredient Name | Lbs/1000 lbs |
|---|---|
| Lodex 15 | 84.88 |
| M-200 | 56.59 |
| Na-caseinate | 42.60 |
| HOSO | 17.76 |
| Ca-caseinate | 14.20 |
| FOS | 10.74 |
| CANOLA oil | 10.65 |
| MCT oil | 7.102 |
| Oat Fiber | 5.667 |
| Fibrim | 4.658 |
| TCP | 2.527 |
| Gum Arabic | 2.218 |
| Na-citrate | 2.100 |
| Mg-phosphate | 1.847 |
| Lecithin | 1.816 |
| K-citrate | 1.300 |
| KCl | 1.300 |
| Carboxymethyl Cellulose | 1.007 |
| MgCl2 | 0.9100 |
| Vit. C | 0.7000 |
| Choline-Cl | 0.6900 |
| Di- Potassium Phos. | 0.3000 |
| UTM/TM | 0.2811 |
| Carnitine | 0.1819 |
| Taurine | 0.1681 |
| Vit. premix | 0.08868 |
| DEK premix | 0.06123 |
| Beta-carotene | 0.00944 |
| Vitamin A | 0.00264 |
| Kl | 0.00020 |

TABLE 6
BOM of a 1.2 Kcal fiber containing product with 20% SPI

| Commodity # | Ingredient Name | Lbs/1000 lbs |
|---|---|---|
| 1302 | Lodex 15 | 84.41 |
| 1313 | M-200 | 56.27 |
| 1980 | Na-caseinate | 31.24 |
| 1734 | HOSO | 17.76 |
| 1970 | Ca-caseinate | 14.20 |
| 1922 | SPI | 12.01 |
| 13736 | FOS | 10.74 |
| 1117 | CANOLA oil | 10.65 |
| 1115 | MCT oil | 7.102 |
| 1918 | Fibrim | 6.964 |
| 12399 | Oat Fiber | 3.766 |
| 1442 | TCP | 2.527 |
| 1336 | Gum Arabic | 2.218 |
| 1430 | Na-citrate | 2.100 |
| 1650 | Mg-phosphate | 1.847 |
| 16973 | Lecithin | 1.816 |
| 1423 | K-citrate | 1.300 |
| 1422 | KCl | 1.300 |
| 1337 | CMC | 1.007 |
| 1418 | MgCl2 | 0.9100 |
| 1201 | Vit. C | 0.7000 |
| 1444 | Choline-Cl | 0.6900 |
| 1426 | Di- Potassium Phos. | 0.3000 |
| 1148 | UTM/TM | 0.2811 |
| 1238 | Carnitine | 0.1819 |
| 1237 | Taurine | 0.1681 |
| 1273 | vit. Premix | 0.08868 |
| 12477 | DEK premix | 0.06123 |
| 1985 | Beta-carotene | 0.00944 |
| 1254 | Vitamin A | 0.00264 |
| 1427 | Kl | 0.00020 |

TABLE 7
BOM of a fiber containing product containing 35% SPI

| Ingredient Name | Lbs/1000 lbs |
|---|---|
| Lodex 15 | 84.41 |
| M-200 | 56.27 |
| Na-caseinate | 25.40 |
| HOSO | 17.76 |
| Ca-caseinate | 14.20 |
| SPI | 21.10 |
| FOS | 10.74 |
| CANOLA oil | 10.65 |
| MCT oil | 7.102 |
| Fibrim | 6.964 |
| Oat Fiber | 3.766 |
| TCP | 2.527 |
| Gum Arabic | 2.218 |
| Na-citrate | 2.100 |
| Mg-phosphate | 1.847 |
| Lecithin | 1.816 |
| K-citrate | 1.300 |
| KCl | 1.300 |
| CMC | 1.007 |
| MgCl2 | 0.9100 |
| Vit. C | 0.7000 |
| Choline-Cl | 0.6900 |
| Di- Potassium Phos. | 0.3000 |
| UTM/TM | 0.2811 |
| Carnitine | 0.1819 |
| Taurine | 0.1681 |
| Vit. premix | 0.08868 |
| DEK premix | 0.06123 |
| Beta-carotene | 0.00944 |
| Vitamin A | 0.00264 |
| Kl | 0.00020 |

The finished products are stored in upright position at room temperature and the thickness of the cream layer during shelf life testing are measured (Table 8). We found that the inclusion of SPI as part of the protein system delayed the onset of creaming and the beneficial effect is a function of SPI level (Table 8).

TABLE 8
Effect of inclusion of SPI on Cream Stability of the 1.2 Kcal fiber
containing product

| | 100% | | |
|---|---|---|---|
| Age of | caseinates | 20% SPI | 35% SPI |
| product | Thickness of | Thickness of | Thickness of |
| (month) | cream (mm) | cream (mm) | cream (mm) |
| 0 | 0.0 | 0.0 | 0.0 |
| 3 | 4.0 | 3.0 | 0.0 |

### EXAMPLE 3

Two fiber containing tube products containing 25% protein, 23% fat and 52% fat calories are prepared using a process described in example 1 including two visits to the pilot plant using various lots of fibers and proteins. Table 9 and 10 showed the BOM of these two formulations.

TABLE 9
BOM of 25% protein calorie fiber containing product made with 100%
caseinate

| INGREDIENT | lb per 1000 lb |
|---|---|
| Maltodextrin-100 | 102.60 |
| Na-caseinate | 55.349 |
| Sucrose | 16.500 |
| Oat fiber | 13.198 |
| HI OLEIC SAFF | 12.570 |
| Ca-caseinate | 8.5643 |
| CANOLA | 7.5360 |
| MCT oil | 5.0160 |
| Fibrim | 2.9944 |
| Mg Phosphate | 2.6670 |
| Nat & art. Vanilla | 2.2500 |
| Lecithin | 1.7600 |
| K CHLORIDE | 1.6556 |
| NA CITRATE | 1.5495 |
| Vanilla Flavor | 1.5000 |
| DCP | 1.3758 |
| Calcium Citrate | 1.2970 |
| Calcium carbonate | 1.2939 |
| K CITRATE | 0.81714 |
| 45% KOH | 0.32200 |
| VITAMIN C | 0.74699 |
| CHOLINE CHLOR | 0.69937 |
| K2PO4 | 0.54951 |
| UTM/TM PREMIX | 0.29973 |
| TAURINE | 0.18753 |
| CARNITINE | 0.16235 |
| VITAMIN PREMIX | 0.10339 |
| Gellan | 0.089921 |
| DEK PREMIX | 0.064159 |
| VITAMIN A | 0.010057 |
| 30% B-CAROTENE | 0.0059945 |
| K IODIDE | 0.0002286 |

TABLE 10
BOM of a 25% protein calorie fiber containing product containing 7% SPI

| | |
|---|---|
| Maltodextrin-100 | 100.0 |
| Na-caseinate | 40.0 |
| M-200 | 20.8 |
| Ca-caseinate | 20.0 |
| HI OLEIC SAFF | 11.6 |
| FOS | 7.15 |
| CANOLA | 6.99 |
| Oat fiber | 4.75 |
| MCT oil | 4.66 |
| Supro 16160 | 4.06 |
| Fibrim | 3.90 |
| K CITRATE | 2.80 |
| Gum Arabic | 1.85 |
| Calcium carbonate | 1.70 |
| Lecithin | 1.03 |
| K CHLORIDE | 1.00 |
| Na Citrate | 0.900 |
| Carboxymethyl Cellulose | 0.838 |
| Mg Phosphate | 0.700 |
| CHOLINE CHLOR | 0.699 |
| UTM/TM PREMIX | 0.380 |
| MG CHLORIDE | 0.380 |
| VITAMIN C | 0.348 |
| VITAMIN PREMIX | 0.203 |
| TAURINE | 0.189 |
| CARNITINE | 0.0800 |
| DEK PREMIX | 0.0422 |
| VITAMIN E | 0.0065 |
| 30% B-CAROTENE | 0.0050 |
| VITAMIN A | 0.0015 |
| K IODIDE | 0.00023 |
| Cr Chloride | 0.00020 |

We measured the cream layer thickness during shelf life (Table 11). We noticed that the SPI formulation has less creaming after 6 months of storage even it does not contain any stabilizer (table 9 and 10). We attribute the improvement in cream stability to inclusion of SPI as part of the protein system.

TABLE 11
Cream layer thickness of two 25% protein calorie fiber container
products

| | 100% caseinate - | 7% SPI - | 100% caseinate - | 7% SPI - |
|---|---|---|---|---|
| | lot 1 | lot 1 | lot 2 | lot 2 |
| time | cream thickness | cream thickness | cream thickness | cream thickness |
| (months) | (mm) | (mm) | (mm) | (mm) |
| 0 | 0 | 0 | 0 | 0 |
| 3 | 3 | 1 | 2 | 2 |
| 6 | Nav | Nav | 4 | 2 |

### EXAMPLE 4

We made two 49%/fat calorie fiber containing products using a process described in example 1. Formula 1 contains 16.7% protein calorie and uses 100% caseinates as its source of protein (table 12) while Formula 2 contains 18% protein calorie and include 20% SPI in its protein system (Table 13).

TABLE 12
BOM of a 100% caseinate fiber containing product containing 49% fat
calorie

| | |
|---|---|
| Maltodextrin-100 | 60.9386820276498 |
| HI OLEIC SAFF | 45.8 |
| Sodium caseinate | 36.9843478260869 |
| Fibrim 300 | 20.1205479452055 |
| Fructose | 17.882368 |
| Calcium Caseinate | 5.62434782608696 |
| CANOLA | 5.4 |
| Lecithin | 2.7 |
| Mg CHLORIDE | 2.3 |
| TCP | 1.52 |
| Na CITRATE | 1.239 |
| vanilla flavor | 1.1 |
| INOSITOL | 0.913876651982379 |
| K CITRATE | 0.826 |
| VITAMIN C | 0.730396475770925 |
| K2HPO4 | 0.666 |
| K CHLORIDE | 0.635 |
| CHOLINE CHLOR | 0.558590308370044 |
| UTM/TM PREMIX | 0.203854625550661 |
| VISCARIN SD-359 | 0.175 |
| CARNITINE | 0.152422907488987 |
| TAURINE | 0.125881057268722 |
| VITAMIN PREMIX | 0.0753303964757709 |
| DEK PREMIX | 0.0629251101321586 |
| 30% B-CAROTENE | 0.00890088105726872 |
| VITAMIN A | 0.0061431718061674 |
| K IODIDE | 0.00013215859030837 |

TABLE 13
BOM of a 20% SPI fiber containing product containing 49% fat calorie

| Ingredient name | In per 1000 lb |
|---|---|
| Maltodextrin 100 | 60.3958700579199 |
| HOS OIL | 45.657 |
| NA CASEINATE | 32.7717391304348 |
| FRUCTOSE | 17.6 |
| Soy Protein Isolate | 10 |
| CA CASEINATE | 6.35869565217391 |
| FIBRIM | 6.01691027069542 |
| CANOLA OIL | 5.643 |
| FOS | 4.19817873128569 |
| OAT FIBER | 3.25358851674641 |
| LECITHIN | 2.7 |
| MG CHLORIDE | 2.3 |
| GUM ARABIC | 1.89792663476874 |
| TCP | 1.52 |
| NA CITRATE | 1.239 |
| Vanilla Flavor | 1.1 |
| INOSITOL | 0.913876651982379 |
| Carboxymethyl | 0.861244019138756 |
| Cellulose | |
| K CITRATE | 0.826 |
| K2HP04 | 0.666 |
| K CHLORIDE | 0.635 |
| CHOLINE CHLORIDE | 0.530660792951542 |
| VITAMIN C | 0.486784140969163 |
| CA CARBONATE | 0.3649 |
| 45% KOH | 0.336222222222222 |
| VISCARIN SD-359 | 0.175 |
| UTM/TM | 0.203854625550661 |
| CARNITINE | 0.152422907488987 |
| TAURINE | 0.125881057268722 |
| VIT. PREMIX | 0.0753303964757709 |
| DEK PREMIX | 0.0629251101321586 |
| VITAMIN E | 0.0272 |
| B-CAROTENE | 0.0089 |
| VITAMIN A | 0.0061431718061674 |
| PYROXIDINE HCL | 0.00149 |
| FOLIC ACID | 0.000245 |
| CR CHLORIDE | 0.000175049597385926 |
| K IODIDE | 0.00013215859030837 |

We measured the cream layer thickness during storage and found that inclusion of SPI delay the onset of creaming (Table 14).

TABLE 14
Cream layer thickness of Glucerna with and without SPI

| Time (months) | 100% caseinate (mm) | 20% SPI (mm) |
|---|---|---|
| 0 time | 0 | 0 |
| 2 months | 3 | 0 |

### EXAMPLE 5

Total of 18 Jevity FOS with various protein systems are made using the method described in method 1. The retorted product were visually inspected and scored based on a 0 to 5 points system. Score of 5 indicates that product exhibits no visible creaming and no signs of protein coagulation. Score 4 indicates that product exhibits less than 2 mm of creaming but has no sign of protein coagulation. Score of 3 indicates that products have greater than 2 mm of creaming but the products are still free of protein coagulation. Score of 2 indicates that there are visible particles, which are likely due to protein coagulation in the products. Score of 1 indicates that the protein aggregates are less than 0.1 cm but they settles so fast that products exhibit wheying at the top of the liquid within 3 days. Score of 0 indicates that protein aggregates are more than 0.1 cm in diameter and product exhibits wheying within 1 day. Product with a score of 1 or less may clog feeding tube and consider functionally unacceptable. Products with a score of less than 3 are not aesthetically unacceptable.

TABLE 15
Protein system

| | | Caseinate | Stability |
|---|---|---|---|
| Whey (%) | Soy (%) | (%) | Score |
| 12.5 | 35 | 47.5 | 5 |
| 25 | 63 | 12 | 1 |
| 12.5 | 52.5 | 35 | 2 |
| 25 | 0 | 75 | 4 |
| 12.5 | 17.5 | 70 | 5 |
| 0 | 70 | 30 | 2 |
| 0 | 35 | 65 | 5 |
| 25 | 31.5 | 43.5 | 2 |
| 0 | 0 | 100 | 3 |
| 25 | 15.8 | 59.2 | 5 |
| 18 | 70 | 12 | 0 |
| 9 | 70 | 21 | 0 |
| 16.7 | 0 | 83.3 | 5 |
| 25 | 47.3 | 72.3 | 1 |
| 0 | 52.5 | 47.5 | 2 |
| 0 | 17.5 | 82.5 | 5 |
| 8.3 | 0 | 91.7 | 4 |
| 12.5 | 35 | 43.5 | 5 |

We claim:

1 A liquid nutritional formula comprising:
a) a protein system which provides at least 16% of the total calories of said formula, in which said protein system contains
i. A source of caseinate protein, present in the quantity of about 40 w/w % to about 95 w/w %, based upon the total protein content of the nutritional, and,
ii. a stabilizing protein selected from the group consisting of vegetable protein and whey protein, in which said stabilizing protein is present in the quantity of about 5 w/w % to about 60 w/w %, based upon the total protein content of the nutritional;
b) a source of fat providing at least 25% of the total calories of said nutritional;
c) a source of carbohydrate providing at least 30% of the total calories of said nutritional, and;
d) at least 8 grams per liter of a source of fiber.

2. The liquid nutritional of claim 1 in which said caseinate protein is selected from the group consisting of sodium caseinate, calcium caseinate and, hydrolyzed caseinate.

3. The liquid nutritional according to claim 1 in which said vegetable protein is soy.

4. The liquid nutritional according to claim 1 in which said stabilizing protein is whey.

5. The liquid nutritional according to claim 1 in which said protein provides from 16% to about 28% of total calories of said nutritional.

6. The liquid nutritional according to claim 1 in which said caseinate is present in the quantity of from about 60 to 85 w/w %, based upon the total protein content of the nutritional.

7. The liquid nutritional according to claim 1 in which said stabilizing protein is present in the quantity of from about 15 to about 40 w/w % based upon the total protein content of the nutritional.

8. The liquid nutritional according to claim 1 in which said fat source provides from about 25% to about 50% of total calories.

9. The liquid nutritional according to claim 1 in which said carbohydrate provides from about 30% to about 60% of total calories.

10. The liquid nutritional according to claim 1 in which said fiber provides a source of fiber selected from the group consisting of soluble fibers and insoluble fibers.

11. The liquid nutritional according to claim 1 in which said source of fiber is selected from the group consisting of gum arabic, carboxymethyl cellulose, guar gum, konjac flour, xanthan gum, alginate, gellan gum, gum acacia, citrus pectin, low and high methoxy pectin, modified cellulose, oat and barley glucans, carrageenan, psyllium, soy polysaccharide, oat hull fiber, pea hull fiber, soy hull fiber, soy cotyledon fiber, sugar beet fiber, cellulose, corn bran and hydrolyzed forms of the listed fibers.

12. The liquid nutritional according to claim 1 in which said nutritional has a caloric density of at least 1 kcal/ml to about 2 kcal/ml.

13. The liquid nutritional according to claim 1 in which said fat source is selected from the group consisting of soy oil, olive oil, marine oil, sunflower oil, high oleic sunflower oil, safflower oil, high oleic safflower oil, fractionated coconut oil, cottonseed oil, corn oil, canola oil, palm oil, palm kernel oil and mixtures thereof

14. The liquid nutritional according to claim 1 having a caloric density of at least 1.2 kcal/ml.

15. A method for reducing creaming in a nutritionally complete liquid formula comprising:
a) Incorporating into said nutritional a source of protein including at least two different proteins,
i. in which one protein is a caseinate protein, present in the quantity of about 45 w/w % to about 85 w/w %, based upon the total protein content of the nutritional,
ii. and a second protein is a stabilizing protein selected from the group consisting of soy protein and whey protein, in which said stabilizing protein is present in the quantity of about 15w/w % to about 55 w/w %, based upon the total protein content of the nutritional.
